(19) United States
(12) Patent Application Publication
(10) Pub. No.: US 2022/0299519 A1
(43) Pub. Date: Sep. 22, 2022

(54) Patch-Sized Apparatus And Method For Use With An Apparel To Detect Seminal Fluid

(71) Applicant: Junell Jilers, Elmore, AL (US)

(72) Inventor: Junell Jilers, Elmore, AL (US)

(21) Appl. No.: 17/203,647

(22) Filed: Mar. 16, 2021

(51) Int. Cl. G01N 33/68; A61F 13/49; A41D 13/00; G01N 21/78

(52) U.S. Cl. CPC G01N 33/689; A61F 13/49; A41D 13/00; G01N 21/78

(57) ABSTRACT

The present invention is a patch-sized apparatus for use with an apparel to detect seminal fluid that includes disposable housing. The disposable housing is removably attached to the apparel. The disposable portion incorporates therein a fluid channel comprising a chemical solution to detect seminal fluid present on the surface of the apparel. The chemical solution includes but not limited to 1-Naphthylphosphate (disodium salt); and prostate-specific antigen (PSA)(A67-B/E3).

### CROSS-REFERENCE TO RELATED APPLICATIONS

None.

### BACKGROUND OF THE INVENTION

The disclosure relates to an embodiment for detecting seminal fluid on various apparel, and more particularly the invention is an embodiment that provides a method for determining whether or not seminal fluid has come in contact with clothing or other accessories and products. The embodiment allows a user to effectively make an assessment whether or not an apparel has come in contact with seminal fluid where certain groups of individuals are at risk.

### DISCUSSION OF THE STATE OF THE ART

Children and other vulnerable individuals are being sexually abused in much higher numbers now more than ever. These individuals often time cannot communicate to others due to their ages or even mental disabilities. Most people who are responsible for these individuals never have an opportunity to know immediately if their love ones have been abused. Historically, victims often have to live with the emotional trauma experienced during the abuse. In an article by StopITNow.Org, it was reported that factors can increase sexual abuse are instances where secrecy is frequently permitted in settings and children being viewed or talked about in adult sexual terms; in fact, it has been reported that child-on-child sexual abuse is on the rise in many areas. Furthermore, in some cases elderly adults are abused at the hands of those who are placed in a position of authority. Accordingly, what is needed in the art is a novel manner of detecting seminal fluid to create a means to provide some form of protection for vulnerable individuals. What is further needed in the art is a novel invention that will allow those responsible for caring for such individuals to accurately obtain evidence of such acts. The proposed embodiment is further ideal because its ultimate purpose is to provide a voice to those who have traditionally not been able to communicate the fact that they have been sexually abused in a manner that will be a welcoming change for the public.

### SUMMARY OF THE INVENTION

According to embodiments illustrated herein, there is provided a patch-sized apparatus and method for use with an apparel to detect seminal fluid.

According to embodiments illustrated herein, there is provided a patch-sized apparatus for use with an apparel to detect seminal fluid that includes a disposable housing. In an aspect, the apparel includes but is not limited to underwear, diapers, wipes, pajamas, shirt, dresses, and bedding accessories. The disposable housing is removably attached to the apparel. The disposable portion incorporates therein a fluid channel comprising a chemical solution to detect seminal fluid present on the surface of the apparel. In an aspect, the chemical solution includes but not limited to 1-Naphthylphosphate (disodium salt); and prostate-specific antigen (PSA) (A67-B/E3).

According to embodiments illustrated herein, there is provided a method to detect the presence of seminal fluid in an apparel. The method includes a step of providing a patch-sized apparatus for use with the apparel. The patch-sized apparatus includes disposable housing. The method includes a step of removably attaching the disposable housing with the apparel. The disposable portion incorporates therein a fluid channel comprising a chemical solution to detect seminal fluid present on the surface of the apparel. In an aspect, the chemical solution comprising: 1-Naphthylphosphate (disodium salt); and prostate-specific antigen (PSA)(A67-B/E3).

Accordingly, one advantage of the present invention is that it communicates the wet status of the apparel to a parent or a caretaker by a color indication.

Accordingly, one advantage of the present invention is that it is easy to use.

Accordingly, one advantage of the present invention is that it can be produced on a large-scale.

The aforementioned features and advantages of the present disclosure may be appreciated by reviewing the following description of the present disclosure, along with the accompanying Figures, wherein like reference numerals refer to like parts.

The objects and advantages of the present invention will become apparent to those skilled in the art when the following description of examples of structure representing the best modes contemplated at the present for practicing the invention is read in conjunction with the accompanying drawing wherein like references numerals refer to like or equivalent parts.

### BRIEF DESCRIPTION OF THE DRAWING FIGURES

The accompanying drawings illustrate several embodiments of the invention and, together with the description, serve to explain the principles of the invention according to the embodiments. It will be appreciated by one skilled in the art that the particular embodiments illustrated in the drawings are merely exemplary and are not to be considered as limiting of the scope of the invention or the claims herein in any way.

Any person with ordinary skills in the art will appreciate that the illustrated element boundaries in the drawings represent an example of the boundaries. In an exemplary embodiment, one element may be designed as multiple elements, or multiple elements may be designed as one element. In an exemplary embodiment, an element shown as an internal component of one element may be implemented as an external component in another and vice versa. Furthermore, the elements may not be drawn to scale.

Various embodiments will hereinafter be described in accordance with the accompanying drawings, which have been provided to illustrate, not limit, the scope, wherein similar designations denote similar elements, and in which:

FIG. 1 illustrates a perspective view of a patch-sized apparatus for use with apparel such as a diaper, in accordance with an embodiment.

FIG. 2 illustrates a perspective view of a patch-sized apparatus for use with apparel such as onesies, in accordance with an embodiment.

FIG. 3 illustrates a perspective view of a patch-sized apparatus for use with apparel such as bedding accessories, in accordance with an embodiment.

FIG. 4 illustrates a perspective view of a patch-sized apparatus for use with apparel such as a frock, in accordance with an embodiment.

FIG. 5 illustrates a flowchart of a method to detect the presence of seminal fluid in apparel, in accordance with an embodiment.

### DETAILED DESCRIPTION OF THE PREFERRED EMBODIMENT

The present disclosure is best understood with reference to the detailed drawings and description set forth herein. Various embodiments have been discussed with reference to the drawings. However, the person skilled in the art will readily appreciate that the detailed descriptions provided herein with respect to the drawings are merely for explanatory purposes, as the apparatus, systems, and methods may extend beyond the described embodiments. For instance, the teachings presented and the needs of a particular application may yield multiple alternate and suitable approaches to implement the functionality of any detail described herein. Therefore, any approach may extend beyond certain implementation choices in the following embodiments.

The term “plurality”, as used herein, is defined as two, or more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

References to “one embodiment”, “at least one embodiment”, “an embodiment”, “one example”, “an example”, “for example”, and so on indicate that the embodiment(s) or example(s) may include a particular feature, structure, characteristic, property, element, or limitation, but not every embodiment or example necessarily include that particular feature, structure, characteristic, property, element, or limitation. Furthermore, repeated use of the phrase “in an embodiment” does not necessarily refer to the same embodiment.

Unless defined otherwise, all technical and scientific terms used herein have the same meaning as commonly understood by one of the ordinary skills in the art to which this invention belongs. Although any method and material similar or equivalent to those described herein can also be used in the practice or testing of the present invention, the preferred methods and materials have been described. All publications, patents, and patent applications mentioned herein are incorporated in their entirety.

It is noted that as used herein and in the appended claims, the singular forms “a”, “and”, and “the” include plural referents unless the context clearly dictates otherwise. In the claims, the terms “first”, “second”, and so forth are to be interpreted merely as ordinal designations; they shall not be limited in themselves. Furthermore, the use of exclusive terminology such as “solely”, “only”, and the like in connection with the recitation of any claim element is contemplated. It is also contemplated that any element indicated to be optional herein may be specifically excluded from a given claim by way of a “negative” limitation. Finally, it is contemplated that any optional feature of the inventive variation(s) described herein may be set forth and claimed independently or in combination with any one or more of the features described herein.

Reference throughout this document to “existing products” refers to any number of traditional products that may come in contact with seminal fluids. Therefore, wherever this specification refers to existing products it will not be limited to any specific product but rather any number of sanitary products and structures may be combined for purposes of the present embodiment. In a non-limiting example, the existing sanitary product can be in reference to a sanitary napkin or tampon but is not limited to these examples.

Headings of sections provided in this patent application and the title of this patent application are for convenience only and are not to be taken as limiting the disclosure in any way.

All references cited herein, including publications, patent applications, and patents, are hereby incorporated by reference to the same extent as if each reference were individually and specifically indicated to be incorporated by reference, and were outlined in its entirety herein.

The recitation of ranges of values herein is merely intended to serve as a shorthand method of referring individually to each separate value falling within the range, unless otherwise indicated herein, and each separate value is incorporated into the specification as if it were individually recited herein.

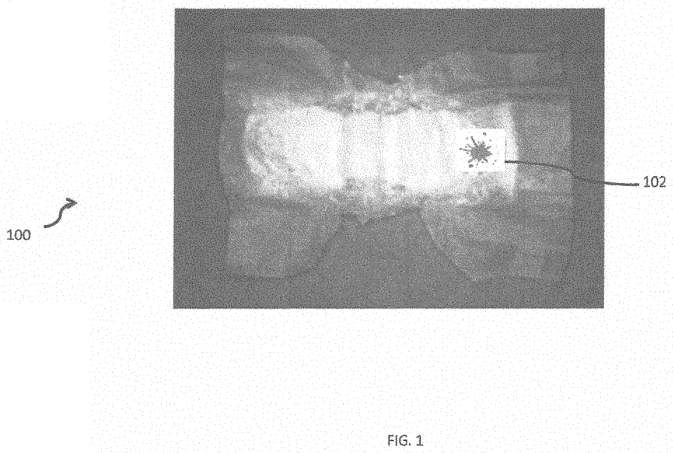

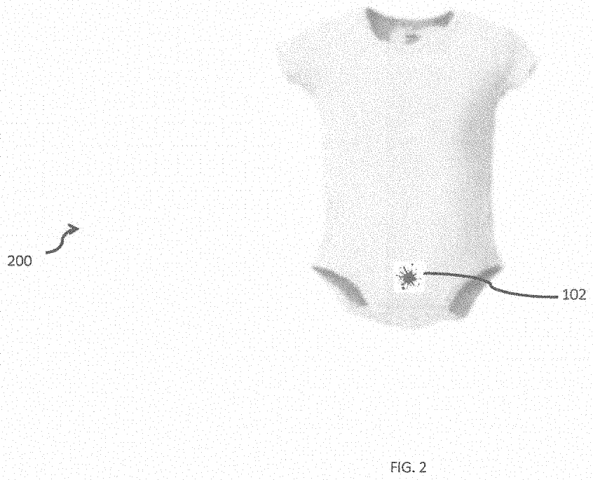

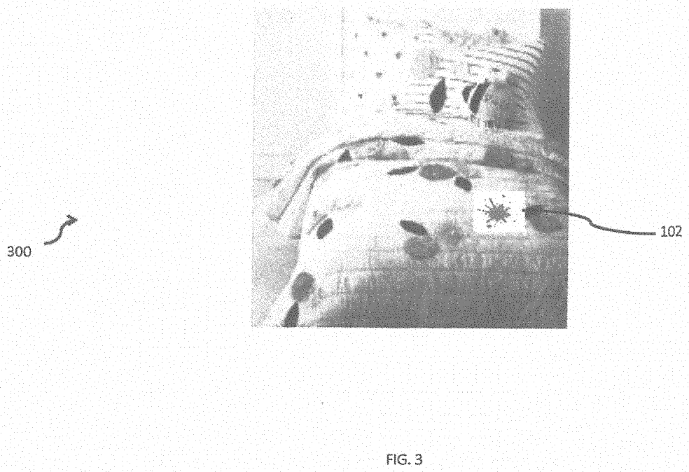

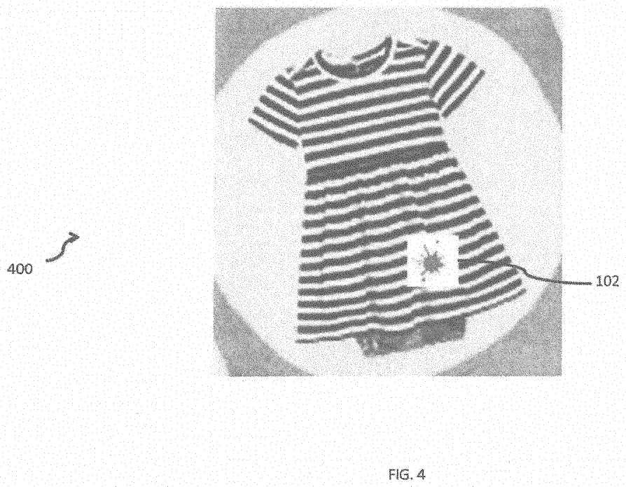

The present invention provides a patch-sized apparatus for use with an apparel to detect seminal fluid that includes disposable housing. In an embodiment, the apparel includes but is not limited to underwear, diapers, wipes, pajamas, shirt, dresses, and bedding accessories. In an embodiment, seminal fluid refers to an organic fluid created to contain spermatozoa which are secreted by the sexual glands and. other sexual organs of a human being. Seminal fluid may include waste products such as urine, or any metabolic waste eliminated through excretion. FIGS. 1-4 illustrate perspective views of a patch-sized apparatus 102 for use with apparel such as a diaper 100, onesies 200, bedding accessories 300, and a frock 400, in accordance with an embodiment. The disposable housing is removably attached to the apparel. The disposable portion incorporates therein a fluid channel comprising a chemical solution to detect seminal fluid present on the surface of the apparel. In an embodiment, the chemical solution includes but not limited to 1-Naphthylphosphate (disodium salt); and prostate-specific antigen (PSA)(A67-B/E3).

1-Naphthyl phosphate (disodium salt) (CAS 2183-17-7) is a histochemical substrate for acid and alkaline phosphatase. 1-naphthol is released upon hydrolyzation. By simultaneous coupling with a diazonium salt, the corresponding azo-dye is formed. Naphthols can also be detected by fluorescence analysis. 1-Naphthyl phosphate acts as a substrate for the determination of prostatic acid phosphatase. The molecular formula for 1-Naphthylphosphate is C₁₀H₇Na₂O₄P.

Prostate-specific antigen, also known as gamma-seminoprotein or kallikrein-3, is a glycoprotein enzyme encoded in humans by the KLK3 gene. PSA is a member of the kallikrein-related peptidase family and is secreted by the epithelial cells of the prostate gland. Anti-PSA Antibody (A67-B/E3) is recommended for the detection of PSA of human origin.

In an embodiment, the present patch-sized apparatus and method may utilize an enzyme-linked immunoabsorption test (ELISA) for the determination of uromucoid present on the apparel. Further, the patch-sized apparatus may incorporate a plurality of textile-based chemical and physical sensors to detect seminal fluid. Examples of chemical and physical sensors include but are not limited to humidity sensors, respiration rate sensors, temperature sensors, and pressure sensors.

According to an embodiment, the disposable housing of the present patch-sized apparatus may include an amniotic fluid detection composition which includes a solvent, a film-forming agent, a plasticizer, wetting agents, and indicators. The solvent may include water, ethyl alcohol, acetone, and N,N-dimethylformamide. The film-forming agent includes pullulan, sodium alginate, ethylcellulose, and sodium carboxymethylcellulose. The plasticizer includes dioctyl phthalate. Wetting agents may include ethylene glycol, glycerol, and glycol ethylene ether. The indicator includes bromothymol blue sodium salt, bromoxylenol blue, sodium alizarine sulfonate, and bromocresol purple. The amniotic fluid detection composition is obtained at the normal temperature, then coating the disposable housing with the amniotic fluid detection composition, and drying the disposable housing to prepare the patch-sized apparatus.

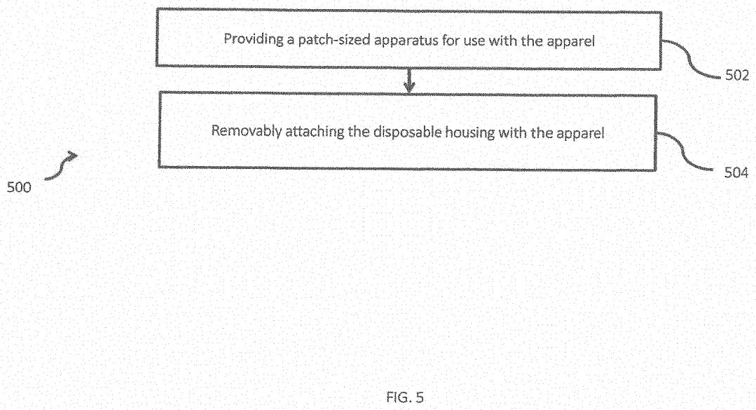

FIG. 5 illustrates a flowchart of a method to detect the presence of seminal fluid in apparel, in accordance with an embodiment. The method includes step 502 of providing a patch-sized apparatus for use with the apparel. The patch-sized apparatus includes disposable housing. The method includes step 504 of removably attaching the disposable housing with the apparel. The disposable portion incorporates therein a fluid channel comprising a chemical solution to detect seminal fluid present on the surface of the apparel. In an aspect, the chemical solution comprising: 1-Naphthylphosphate (disodium salt); and prostate-specific antigen (PSA) (A67-B/E3).

Thus, the present patch-sized apparatus communicates the wet status of the apparel to a parent or a caretaker by a color indication. The disposable housing of the patch-sized apparatus changes color in response to temperature change, pH change, and/or the presence of water/fluid.

While embodiments of the present invention have been illustrated and described, it will be clear that the present invention is not limited to these embodiments only. Numerous modifications, changes, variations, substitutions, and equivalents will be apparent to the person skilled in the art, without departing from the scope of the invention, as described in the claims.

What is claimed is:

1. A patch-sized apparatus for use with an apparel to detect seminal fluid, comprising:
a disposable housing removably attached with the apparel, wherein the disposable portion incorporates therein a fluid channel comprising a chemical solution to detect seminal fluid present on a surface of the apparel.

2. The patch-sized apparatus as claimed in claim 1, wherein the chemical solution comprising: 1-Naphthylphosphate (disodium salt); and prostate-specific antigen (PSA)(A67-B/E3).

3. The patch-sized apparatus as claimed in claim 1, wherein the apparel comprising: underwear, diapers, wipes, pajamas, shirt, dresses, and bedding accessories.

4. A method to detect presence of seminal fluid in apparel, comprising:
providing a patch-sized apparatus for use with the apparel, wherein the patch-sized apparatus comprises a disposable housing; and
removably attaching the disposable housing with the apparel, wherein the disposable portion incorporates therein a fluid channel comprising a chemical solution to detect seminal fluid present on a surface of the apparel.

5. The method as claimed in claim 4, wherein the chemical solution comprising: 1-Naphthylphosphate (disodium salt); and prostate-specific antigen (PSA)(A67-B/E3).

6. The method as claimed in claim 4, wherein the apparel comprising: underwear, diapers, wipes, pajamas, shirt, dresses, and bedding accessories.
